(19) United States
(12) Patent Application Publication
(10) Pub. No.: US 2019/0314188 A1
(43) Pub. Date: Oct. 17, 2019

(54) WEARABLE SUPPORTS FOR URINE COLLECTION DEVICES

(71) Applicant: Joel Kwan Barrientos, Centralia, IL (US)

(72) Inventor: Joel Kwan Barrientos, Centralia, IL (US)

(21) Appl. No.: 16/382,685

(22) Filed: Apr. 12, 2019

Related U.S. Application Data
(60) Provisional application No. 62/656,520, filed on Apr. 12, 2018.

(51) Int. Cl. A61F 5/44; A61M 25/10; A61F 5/453

(52) U.S. Cl. CPC A61F 5/4408; A61M 2209/088; A61F 5/453; A61M 25/10181

(57) ABSTRACT

A urine collection system includes a urine collection device and a suspension system. The urine collection device includes a catheter and a urine bag. The suspension system includes a waist band, an extension strap depending from the waist band, and a thigh band attached to the strap. The length of the extension strap can be made adjustable either by proving the strap in two sections releasably attached to each other, or allowing the extension strap to fold over itself. An auxiliary thigh band can be provided in some embodiments. A pair of securing straps is provided on the thigh band for securing the urine bag. A fastener is provided on the extension strap for securing the catheter and for resisting pulling by the urine bag. The catheter further includes a branch, which, along with the fastener, resists the downward motion of the catheter. At least the waist band and the thigh bands can be provided with non-slip elements to help secure the system on the users' body.

### FIELD

The present invention relates generally to urine collection devices, and more particularly to urine collection devices with wearable supports for incontinent patients.

### BACKGROUND

This section provides background information related to the present disclosure which is not necessarily prior art.

A conventional urine collection device includes an external or internal catheter (such as a Foley catheter) communicating with the subject's urethra, and a urine bag connected to the catheter for collecting urine. To enhance the subject's mobility the urine bag can be secured to one of the patient's thighs. The urine collection device is thus hidden under the clothing of the patient so that the patient can engage in daily activities without embarrassment.

Conventional urine collection devices, however, can cause discomfort to the patient. If not properly supported, the urine bag tends to swing back and forth across the patient's thigh as the patient moves, impeding free movement of the patient. Moreover, as more urine is collected, the weight of urine bag can cause it to slide, applying a pulling force that is at best uncomfortable for the subject, and in the case of an internal catheter, can forcibly pull the catheter from the subject's urethra, causing severe trauma and pain to the subject.

Attempts have been made to provide more secure support for urine collection device discomfort and embarrassment, and in many cases pain and trauma are still common side effects of using a mobile urine collection system.

### SUMMARY

This section provides a general summary of the disclosure, and is not a comprehensive disclosure of its full scope or all of its features.

Embodiments of the present invention provide for more secure support for urine collection system with reduced risk to the user. In one preferred form, a urine bag suspension system for carrying a urine collection device is provided. The urine bag suspension system includes a waist band, a urine bag support suspended from the waist band for carrying a urine bag of the urine collection device, and a fastener. The fastener is disposed at the urine bag support for engaging the catheter of the urine collection device to resist pulling it from communication with the subject's urethra.

In another preferred form, a urine bag suspension system for carrying a urine collection device is provided. The urine collection device includes a catheter and a urine bag. The catheter has an engaging end removably engaging the urine bag. The urine bag suspension system includes a waist band, an extension strap suspended from the waist band, and a bag carrier for carrying the urine bag. The extension strap is substantially vertically suspended from the waist band and is connected to about the midway of an upper edge of the bag carrier. The bag carrier includes a pair of securing straps.

In still another preferred form, a wearable support for a urine collection system comprising a catheter and a length of tubing for connection to a collection device is provided. The support comprises a waist band adapted to be worn around the user's waist, a strap depending from the waist band, and a thigh band attached to the strap, adapted to be worn around the user's thigh. The support further comprises at least one fastener on the thigh band for engaging and supporting the length of tubing, and at least one fastener on the strap for engaging the catheter to resist pulling of the tubing, via the fasteners on the thigh band.

Various embodiments of the invention provide more secure support for the urine bags in urine collection systems to resist movement of the bag and attendant discomfort to the subject. Various embodiments of the invention also engage the catheter to resist movement of the catheter and resulting discomfort and trauma to the subject. Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of one embodiment of a urine collection system in accordance with the teachings of the present disclosure;

FIG. 2 is a perspective view of the urine collection system of FIG. 1, wherein the urine collection device is disassembled from a wearable suspension system therefore;

FIG. 3 is a perspective view of a second embodiment of a urine collection system in accordance with the teachings of the present disclosure;

FIG. 4 is a perspective view of the urine collection system of FIG. 3, wherein the urine collection device is disassembled from a wearable suspension system therefore;

FIG. 5 is a perspective view of a first alternate embodiment of the urine collection system of FIGS. 1 and 2, showing an auxiliary thigh band;

FIG. 6A is a perspective view of a second alternate embodiment of the urine collection system of FIGS. 1 and 2, showing an adjustable extension strap strap;

FIG. 6B is a side elevation view of the second alternate embodiment, showing details of the adjustment mechanism for the adjustable extension strap;

FIG. 7A is a perspective view of a third alternate embodiment of the urine collection system of FIGS. 1 and 2, showing an adjustable extension strap strap;

FIG. 78 is a side elevation view of the third alternate embodiment, showing details of the adjustment mechanism for the adjustable extension strap; and

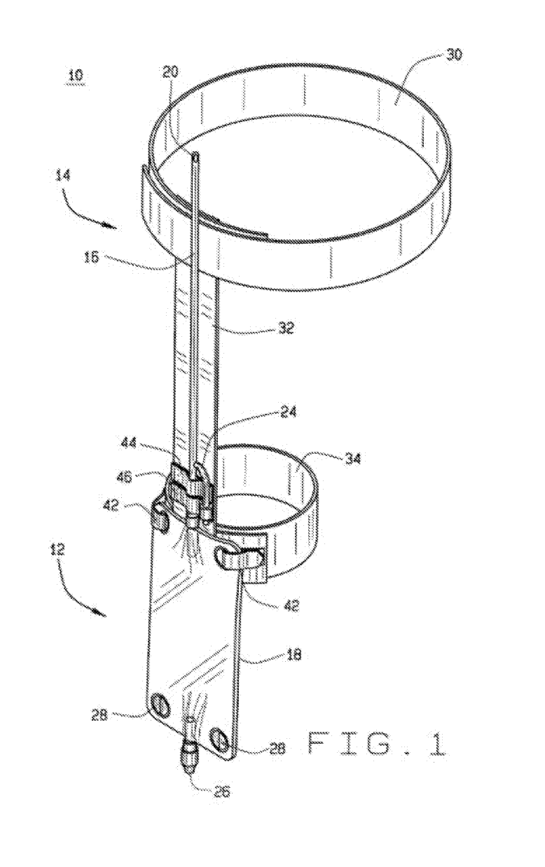

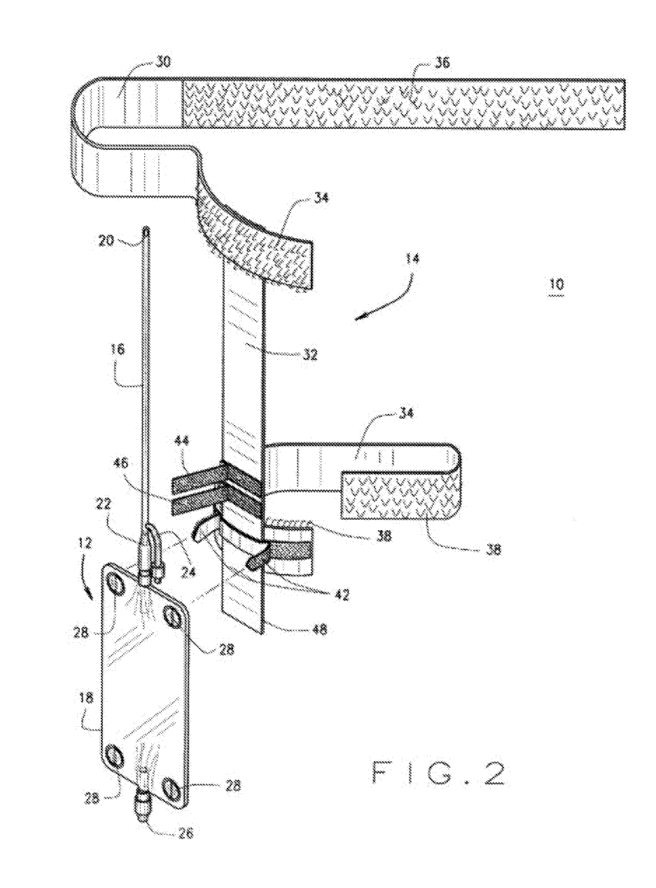

FIG. 8 is a perspective view of a fourth alternate embodiment of the urine collection system of FIGS. 1 and 2, showing an improved waist band and thigh strap.

Corresponding reference numerals indicate corresponding parts throughout the several views of the drawings.

### DETAILED DESCRIPTION

Example embodiments will now be described more fully with reference to the accompanying drawings.

The structure of a urine collection system in accordance with the present disclosure is now described in greater detail. The following description of the illustrated example is merely exemplary in nature and is in no way intended to limit the invention, its application, or uses.

The structure of a urine collection system in accordance with the present disclosure is now described in greater detail. The following description of the illustrated example is merely exemplary in nature and is in no way intended to limit the invention, its application, or uses.

Referring to FIGS. 1 and 2, one embodiment of a urine collection system in accordance with the teachings of the present disclosure is illustrated and generally indicated by reference numeral 10. The urine collection system 10 includes a urine collection device 12 and a wearable support or a wearable suspension system 14. The urine collection device 12 includes a catheter 16 and a urine bag 18. The catheter 16 has an inlet end 20 for communicating with the urethra of the subject, and an outlet end 22 for engaging the urine bag 18.

The catheter 16 can be a Foley catheter, which is adapted to be inserted into the urethra of the patient, or a Texas catheter, which is to be connected to the penis of a male patient via a condom-like envelope, or any other type of internal or external urine collection catheter. In any case, the catheter 16 usually includes a branch 24 adjacent to the outlet end 22, the function of which will be described later.

The urine bag 18 has a discharge outlet 26 at the opposite end of the bag from the connection to the outlet end 22 of the catheter 16 for emptying the urine bag 24. In most applications the bag 18 is mounted generally vertically, with the connection to the outlet end 22 of the catheter at the upper end of the bag, and the discharge outlet 26 at the lower end. The urine bag 18 preferably has a generally rectangular shape with four corners and four eyelets 28, one at each of its four corners for mounting the bag.

The suspension system 14 is used to support and carry the urine collection device 12 on the subject, and includes a waist band 30, an extension strap 32 depending from the waist band 30, and a thigh band 34 attached adjacent the free end of the extension strap 32. The waist band 30 could be a continuous loop, but it is preferably in the form of a belt for encircling the entire waist of the subject. The waist band 30 could also be in the form of a strip to be attached to a piece of clothing around the subject's waist without completely encircling the subject's waist. In either case, the waist band 30 can be provided with a fastener 36 to make it easy for the subject to put the suspension system 14 on, and take it off. In the illustrated example, the fastener 36 includes an elongated band with mating fastener elements. The mating fastening elements are preferably mating patches of a hook-and-loop style fastener, such as Velcro® fastener, to allow easy attachment and detachment. However, any conventional fasteners, such as buckles, buttons, or snaps, could be used for securing the waist band 30 around the subject's waist or to the subject's clothing could be used without departing from the spirit of the present disclosure.

The thigh band 34 is generally parallel to the waist band 30 and is sufficiently long to surround the subject's thigh. The thigh band 34 could be a continuous loop, but it is preferably in the form of a belt a belt for encircling the entire thigh of the subject, The thigh band 34 can be provided with a fastener 38 to make it easy for the subject to put the suspension system 14 on, and take it off. In the illustrated example, the fastener 38 includes an elongated band with mating fastener elements. The mating fastening elements 38 are preferably mating patches of a hook-and-loop style fastener, such as Velcro® fastener, to allow easy attachment and detachment. However, any conventional fasteners, such as buckles, buttons, or snaps, could be used for securing the thigh band 34 around the subject's thigh could be used without departing from the spirit of the present disclosure.

A pair of securing straps 42 is provided on the thigh band 34 adjacent to the extension strap 32. The securing straps 42 are positioned to engage an adjacent opening 28 at the top corners of the urine bag 18 to secure the urine bag 18 on the thigh band 34. It should be noted that while the securing straps 42 are shown to be provided on the thigh band 34 in the illustrated example, the securing straps 42 can be provided on the extension strap 32, instead of thigh band 34, as long as the securing straps 42 can be positioned to engage the adjacent openings 28 of the urine bag 18 to secure the urine bag 18 to the suspension system 14. Because the thigh band generally does not change in dimension, the corners of the bag 18 are held in a substantially fixed distance, preventing crushing of the bag, which could tend to apply a back pressure to the catheter.

The extension strap 32 extends perpendicularly from the waist band 30 a sufficient length so that when the waist band is secured at the subject's wait, the strap extends down along the side of the subject sufficiently such that the thigh band 34 can be attached to the thigh of the subject. A pair of fasteners 44 and 46 are disposed at the extension strap 32 for positioning and securing the catheter 16. The fasteners 44 and 46 are disposed adjacent to the outlet end 22 and below the branch 24. The branch 24 provides for attachment to a pump device, which can be used to inflate a balloon that may be disposed at the end of the catheter 16 for anchoring the catheter within the subject's urethra. The engagement between the fasteners 44 and the branch 24 resists movement of, the catheter 16 due to pulling of the bag from movement by the subject or from the increasing weight of urine that is collected in the bag. Therefore, the catheter 16 remains in a “slack” or “no tension” state despite the increased weight of the urine bag 18 and no pulling force is exerted on the urethra of the patient to cause pain. The fasteners 44 and 46 include fastening means, such as mating patches of a hook-and-loop style fastener, Velcro®, although some other type of fastening means could be used.

A panel 48 is provided at a lower end of the extension strap 32 to separate and insulate the patient's thigh from the urine bag 18. The panel 48 can be of any shape and size and does not have to be an integral part of the extension strap 32 as shown in the illustrated example, as long as the panel 48 provides proper separation between the urine bag 18 and the thigh to improve comfort. The panel 48 can be of sufficient size and length so that a second pair of securing straps or a second thigh band can be attached to the panel 48 to secure the lower end of the urine bag 18. Alternatively, the panel 48 can be in a form of a pouch to receive the urine bag 18 therein.

The waist band 30 and the extension strap 32 are preferably made of a flexible washable material so that the construction of the suspension system 14 is not unduly distorted due to the increased weight of the urine bag 18. Some degree of stretch is desirable, however, so that the waist band 30 and the extension strap 32 accommodate movements of the wearer without pulling or pinching the wearer, or causing tugging on the catheter. It has been found that a woven elastic latex free material works well for this purpose, and a preferred material is one that is 75% polyester, 17% nylon, and 8% spandex. While this material should stretch the accommodate the movements of the wearer, it preferably does not significantly stretch under the weight of the collection system, even when full.

With the construction of the present urine collection system 10, the urine collection device 12 can be easily disassembled from the suspension system 14 for cleaning or emptying purposes.

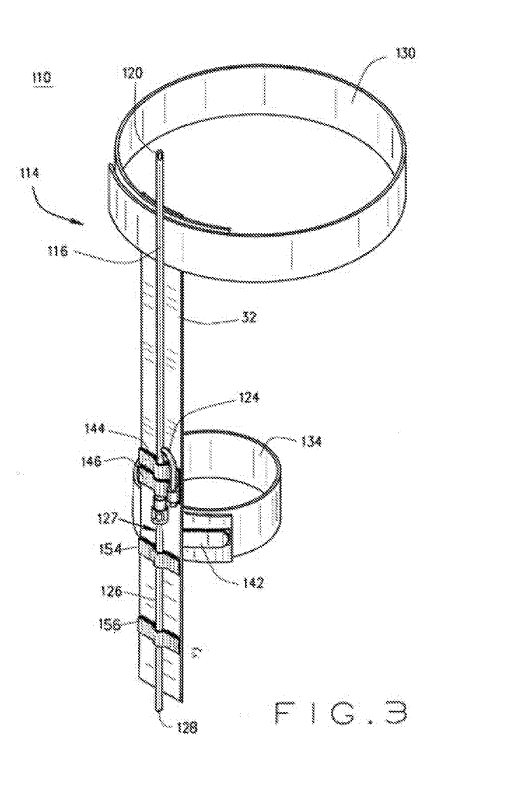

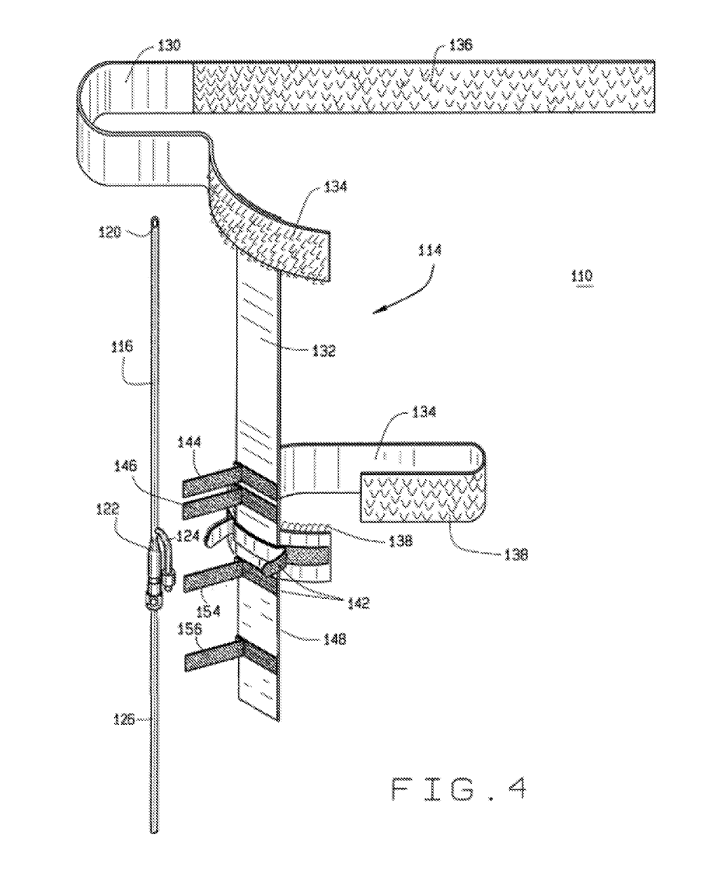

Referring to FIGS. 3 and 4, a second embodiment of a urine collection system in accordance with the teachings of the present disclosure is illustrated and generally indicated by reference numeral 110. The urine collection system 110 includes a catheter 116 and a wearable support or a wearable suspension system 114. The urine collection system further includes a length of tubing 126 that may be secured to the wearable support 114. The catheter 116 has an inlet end 120 for communicating with the urethra of the subject, and an outlet end 122 for engaging the inlet in the upper end 127 of the length of tubing 126.

The catheter 116 can be a Foley catheter, which is adapted to be inserted into the urethra of the patient, or a Texas catheter, which is to be connected to the penis of a male patient via a condom-like envelope, or any other type of internal or external urine collection catheter. In any case, the catheter 116 usually includes a branch 124 adjacent to the outlet end 122, the function of which will be described later.

The urine tubing 126 has a discharge outlet at its lower end for connection to a urine collection bag (not shown). In most applications the length of tubing 126 is mounted generally vertically, with the connection to the outlet end 122 of the catheter at the first inlet end of the tubing 127, and the discharge outlet 128 at the lower end. The urine tubing 126 preferably carries urine from the catheter to a urine collection bag that may be mounted to a stationary patient bed, or to a moveable support such as a stand on wheels. The urine collection apparatus worn by a patient provides for securing tubing of urine collection devices, and may be used with a large Foley catheter urine bag, or with a Suprapubic catheter or cystostomy catheter. Such a catheter is inserted in the lower abdomen above the pubic bone directly into the urinary bladder.

The suspension system 114 is used to support the urine collection components on the subject, and includes a waist band 130, an extension strap 132 depending from the waist band 130, and a thigh band 134 attached adjacent the free end of the extension strap 132. The waist band 130 could be a continuous loop, but it is preferably in the form of a belt for encircling the entire waist of the subject. The waist band 130 could also be in the form of a strip to be attached to a piece of clothing around the subject's waist without completely encircling the subject's waist. In either case, the waist band 30 can be provided with a fastener 136 to make it easy for the subject to put the suspension system 114 on, and take it off. In the illustrated example, the fastener 136 includes an elongated band with mating fastener elements. The mating fastening elements are preferably mating patches of a hook-and-loop style fastener, such as Velcro® fastener, to allow easy attachment and detachment. However, any conventional fasteners, such as buckles, buttons, or snaps, could be used for securing the waist band 130 around the subject's waist or to the subject's clothing could be used without departing from the spirit of the present disclosure.

The thigh band 134 is generally parallel to the waist band 130 and is sufficiently long to surround the subject's thigh. The thigh band 134 could be a continuous loop, but it is preferably in the form of a belt a belt for encircling the entire thigh of the subject, The thigh band 134 can be provided with a fastener 138 to make it easy for the subject to put the suspension system 114 on, and take it off. In the illustrated example, the fastener 138 includes an elongated band with mating fastener elements. The mating fastening elements 138 are preferably mating patches of a hook-and-loop style fastener, such as Velcro® fastener, to allow easy attachment and detachment. However, any conventional fasteners, such as buckles, buttons, or snaps, could be used for securing the thigh band 134 around the subject's thigh could be used without departing from the spirit of the present disclosure.

A pair of securing straps 142 is provided on the thigh band 134 adjacent to the extension strap 132. The securing straps 142 are adapted to engage an opening in the top corners of a urine bag where a urine bag may be used, to secure the urine bag on the thigh band 134. The extension strap 132 extends perpendicularly from the waist band 130 a sufficient length so that when the waist band is secured at the subject's wait, the strap extends down along the side of the subject sufficiently such that the thigh band 134 can be attached to the thigh of the subject. A pair of fasteners 144 and 146 are disposed at the extension strap 132 for positioning and securing the catheter 116. The fasteners 144 and 146 are disposed adjacent to the outlet end 122 and below the branch 124. The branch 124 provides for attachment to a pump device, which can be used to inflate a balloon that may be disposed at the end of the catheter 116 for anchoring the catheter within the subject's urethra. The engagement between the fasteners 144 and the branch 124 resists movement of the catheter 116 due to pulling of the portion of the urine carrying tubing 128 that is not secured to the support. Therefore, the catheter 116 remains in a “slack” or “no tension” state when the tubing 128 is accidentally or inadvertently pulled as a result of movement of the patient or the urine collection bag that is connected to the tubing 128. The fasteners 144 and 146 include fastening means, such as mating patches of a hook-and-loop style fastener, Velcro® although some other type of fastening means could be used.

A panel 148 is provided at a lower end of the extension strap 132 to secure the length of tubing 126 to the patient's leg. The panel 148 can be of any shape and size and does not have to be an integral part of the extension strap 132 as shown in the illustrated example, as long as the panel 148 provides proper separation between the length of urine tubing 126 and the thigh to improve comfort and provide support for the tubing 126. The panel 148 can be of sufficient size and length so that a second pair of securing straps 154 and 156 can be attached to the panel 148 to secure the lower end of the urine tubing 126. The second pair of securing straps 154 and 156 provide for supporting the length of tubing 126, such that movement of a urine collection bag in connection with the end 128 of the tubing 126 does not affect the catheter 116 or cause discomfort to the patient. Likewise, securing straps 154 and 156 sufficiently stabilize the tubing 126 to permit the patient to move relative to a urine collection bag attached to the end of the tubing 128, such that accidental entanglement of the tubing caused by the patient's movement does not result in a force applied to the catheter 116 or cause discomfort to the patient. In the embodiment illustrated in FIGS. 3 and 4, the panel 148 extends a minimum length of at least 4 inches to provide for at least one securing strap 156 spaced about three inches below the thigh band 134. A second thigh band (not shown) may also be attached to the panel 148 to secure the lower end of the panel 148 to the patient's leg, to further stabilize the panel and tubing 126.

Accordingly, the exemplary embodiment shown in the Figures provides a wearable support 110 for a urine collection catheter 116 engagingly receiving a length of collection tubing 126. The wearable support includes the waist band 130 that is adapted to be worn around the user's waist, and the extension strap 132 depending from the waist band 130. The thigh band 134 on the wearable support 110 is attached to the extension strap 132, and is adapted to be worn around the user's thigh. At least one fastener 146 on the extension strap 132 is included for engaging the catheter 116 to resist downward movement of the catheter 116 relative to the extension strap 132. At least one fastener 156 on the lower end portion of the extension strap is included for engaging and securing a portion of collection tubing 126, to resist movement of the collection tubing 126 such that movement of the catheter 116 relative to the extension strap 132 is resisted. The at least one fastener on the extension strap engages the catheter 116 below a branch 124 to resist downward motion of the catheter 116. The at least one fastener 156 on the lower end portion of the extension strap supports the collection tubing 126 in a manner that resists movement of the secured portion of tubing 126 and the catheter 116 relative to the extension strap 132, when an unsecured portion of the collection tubing 128 is being pulled. The thigh band 134 also supports the extension strap 132 in a manner that resists movement of the extension strap 132 relative to the patient when an unsecured portion of the collection tubing 128 is being pulled.

The waist band 130 and the extension strap 132 are preferably made of a washable, non-stretchable material so that the construction of the suspension system 114 is not significantly distorted by movement of the patient.

With the construction of the present urine collection system 110, the urine collection device 112 can be easily disassembled from the suspension system 114 for cleaning or emptying purposes.

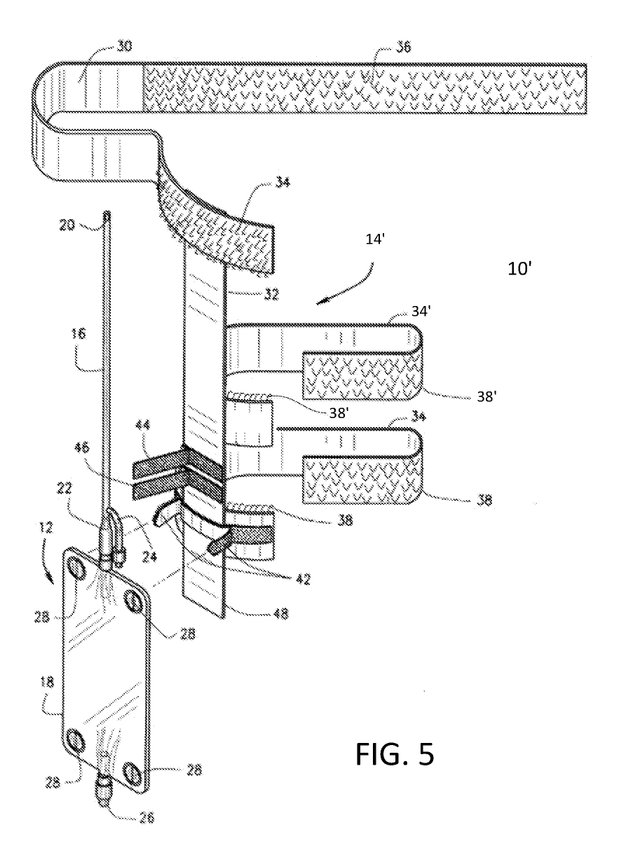

A first alternate embodiment of the urine collection system of FIGS. 1 and 2, indicated generally as 10′, is shown in FIG. 5. The system 10′ is similar to system 10 described above and shown in FIGS. 1 and 2, and corresponding parts are identified with corresponding reference numerals. However system 10′ differs from system 10 by providing a suspension system 14′ with an auxiliary thigh band 34′ extending from the extension strap 32, preferably positioned so that it is vertically above the primary thigh band 34, when the system is worn by a patient. However the auxiliary thigh band 34′ could be provided below the primary thigh band 34, if desired.

The auxiliary thigh band 34′ can be provided with a fastener 38′ to make it easy for the subject to put the suspension system 14 on, and take it off. In the illustrated example, the fastener 38 includes an elongated band with mating fastener elements. The fastener 38 is preferably mating patches of a hook-and-loop style fastener, such as Velcro® fastener, to allow easy attachment and detachment. However, any conventional fasteners, such as buckles, buttons, or snaps, could be used for securing the auxiliary thigh band 34′ around the subject's thigh could be used without departing from the spirit of the present disclosure.

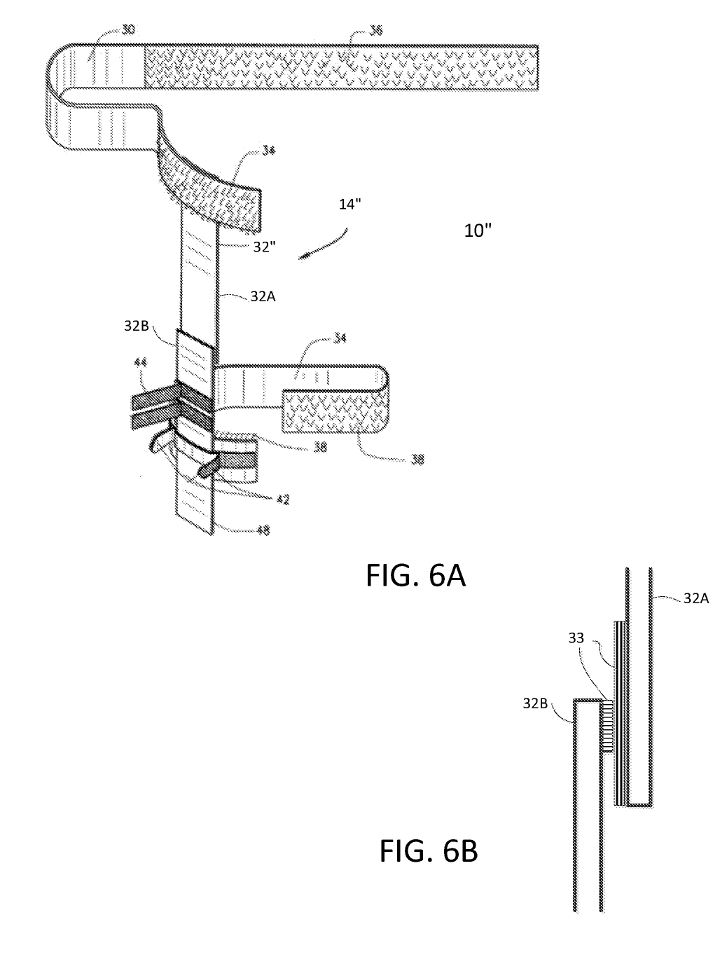

A second alternate embodiment of the urine collection system of FIGS. 1 and 2, is indicted generally as 10″ in FIGS. 6A and 6B. The system 10″ is similar to system 10 described above and shown in FIGS. 1 and 2, and corresponding parts are identified with corresponding reference numerals. However system 10″ differs from system 10 by providing a suspension system 14″ with an adjustable extension strap 32″. As shown in FIGS. 6 and 6A the extension strap 32′ comprises two sections 32A and 32B, which are preferably releasably joined by mating fastener elements. The fastener elements 33 are preferably mating patches of a hook-and-loop style fastener, such as Velcro® fastener, to allow easy attachment and detachment. However, any conventional fasteners, such as buckles, buttons, or snaps, could be used for securing the sections 32A and 32B together, at an appropriate overall length for the extension strap 32″ that is appropriate for the particular user, without departing from the spirit of the present disclosure.

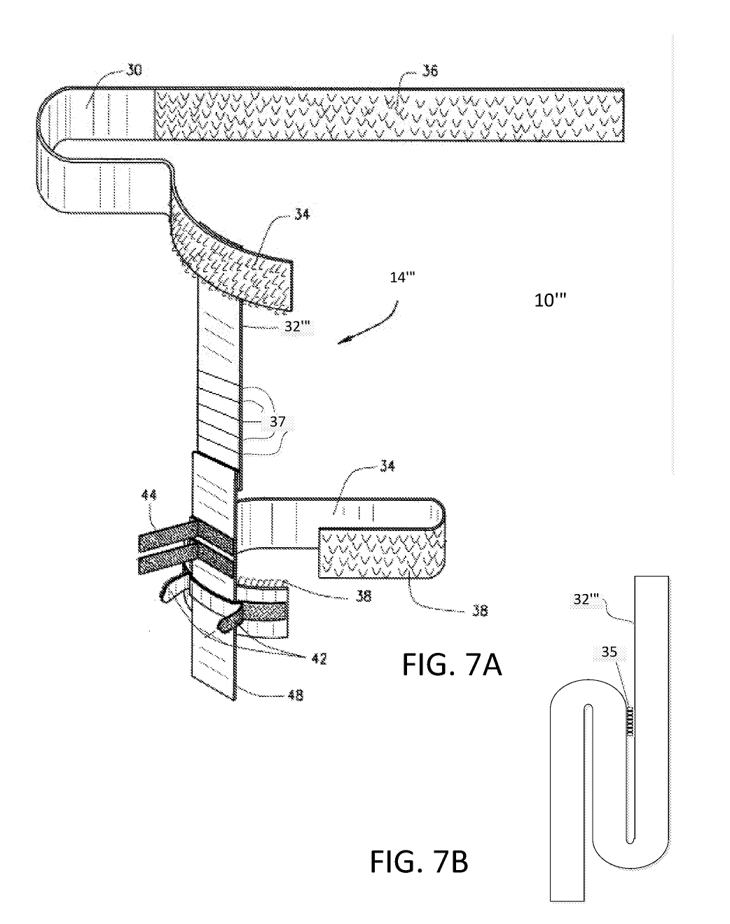

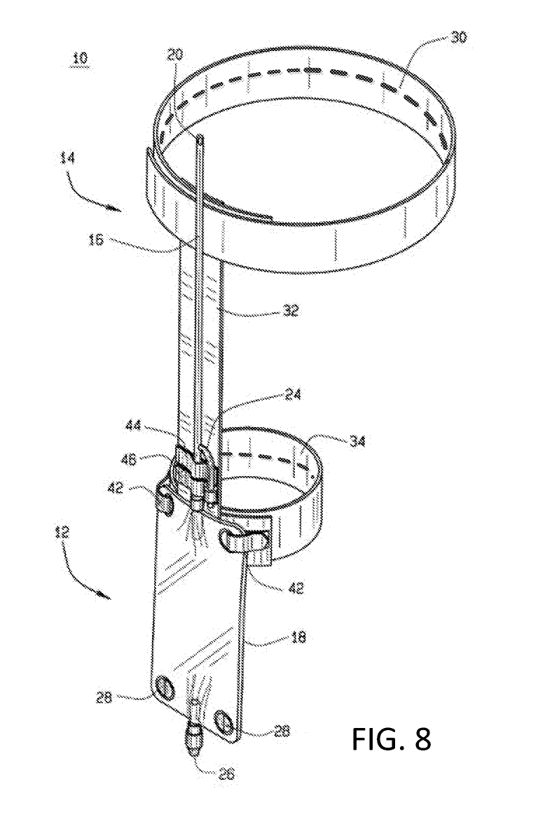

A third alternate embodiment of the urine collection system of FIGS. 1 and 2, is indicted generally as 10′″ in FIGS. 7A and 7B. The system 10′″ is similar to system 10 described above and shown in FIGS. 1 and 2, and corresponding parts are identified with corresponding reference numerals. However system 10′″ differs from system 10 by providing a suspension system 14′″ with an adjustable extension strap 32′″. As shown in FIGS. 7 and 7A the extension strap 32′″ comprises a flexible section that can be looped over itself, and re releasably secured by mating fastener elements. The fastener elements 35 are preferably mating patches of a hook-and-loop style fastener, such as Velcro® fastener, to allow easy attachment and detachment. However, any conventional fasteners, such as buttons, or snaps, could be used for securing the extension strap 32′″ to itself, at an appropriate overall length for the extension strap 32′″ that is appropriate for the particular user, without departing from the spirit of the present disclosure. The strap 32′″ can be provided with indicia 37, which could be lines, colored zones, and labeled segments for facilitating the adjustment of the extension strap to its proper length.

A fourth alternate embodiment of the urine collection system of FIGS. 1 and 2, is indicated generally as 10″″ in FIG. 8. The system 10″″ is similar to system 10 described above and shown in FIGS. 1 and 2, and corresponding parts are identified with corresponding reference numerals. However system 10″″ differs from system 10 by providing anti-slip means in at least one of the waist band and thigh strap. As shown in FIG. 8, such anti-slop means is provided both on the waistband 30 and on the thigh strap 34. This anti-slip means is preferably a non-slip element such as silicone bands 31 woven into or applied on the surfaces of the waist band 30 and thigh band 34 (and the auxiliary thigh band 34′ is one is provided). Of course, other materials could be used, although it is preferably a non-slip, non-stick, hypoallergenic material.

It should be understood that the improvements of the second, third, and fourth alternate embodiments can be combined. In particular the auxiliary thigh band 34′ of the second alternate embodiment could be incorporated into the system of the second, third, and fourth embodiment. Similarly, the anti-slip means can be incorporated into any of the first, second, or third alternate embodiments. The auxiliary thigh band 34 and the antislip bands 31 are particularly useful when used together in the second or third alternate embodiments, as they provide additional security against the adjustment of the extension band 32, particular where the band is formed in two pieces as in the second alternate embodiment.

The description of the invention is merely exemplary in nature and, thus, variations that do not depart from the gist of the invention are intended to be within the scope of the invention. Such variations are not to be regarded as a departure from the spirit and scope of the invention.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A urine bag suspension system for carrying a urine collection device including a catheter and a urine bag, the catheter having an engaging outlet end removably engaging the urine bag, and a branch depending therefrom providing for attachment of the branch to a pump device, the urine bag suspension system comprising: a waist band; a urine bag support suspended from the waist band for carrying a urine bag, wherein the urine bag support comprises an extension strap and first and second thigh bands attached to the extension strap, the first and second thigh bands being positioned on the extension strap relative to the waist band so as to wrap around the thigh of a patient; a pair of securing straps attached to the first thigh band and the extension strap, each securing strap comprising a fastener portion that is configured to be received through an opening in a top corner of a urine collection bag, and to engage a mating portion on the thigh band, to thereby secure the urine bag to the suspension system; and a first fastener positioned on the extension strap above the thigh band, the first fastener being configured to engage the catheter below the branch only, to secure the catheter without securing the branch such that the first fastener resists downward movement of the catheter and provides for attachment of the branch to a pump device; a second fastener positioned on the extension strap above the thigh band and below the first fastener, the second fastener being configured to engage the catheter above the outlet end of the catheter and below the branch of the catheter; whereby the urine bag suspension system is free of fasteners above the branch in the catheter, such that disengaging the first and second fasteners positioned below the branch provides for disassembly of the suspension system from the catheter for cleaning purposes.

2. The urine bag suspension system according to claim 1 further comprising an anti-slip element disposed on the inside of at least one of the waist band and the thigh band.

3. The urine bag suspension system according to claim 2 wherein the anti-slip element comprises silicone material.

4. A urine bag suspension system for carrying a urine collection device including a catheter and a urine bag, the catheter having an engaging outlet end removably engaging the urine bag, and a branch depending therefrom providing for attachment of the branch to a pump device, the urine bag suspension system comprising: a waist band; a urine bag support suspended from the waist band for carrying a urine bag, wherein the urine bag support comprises an extension strap, the extension strap comprising an upper section suspended from the waist band, and a separate lower section adjustably, releasably securable to the upper section, and a thigh band attached to the lower portion of the extension strap, the thigh band being positioned on the extension strap relative to the waist band so as to wrap around the thigh of a patient; a pair of securing straps attached to the thigh band and the extension strap, each securing strap comprising a fastener portion that is configured to be received through an opening in a top corner of a urine collection bag, and to engage a mating portion on the thigh band, to thereby secure the urine bag to the suspension system; and a first fastener positioned on the extension strap above the thigh band, the first fastener being configured to engage the catheter below the branch only, to secure the catheter without securing the branch such that the first fastener resists downward movement of the catheter and provides for attachment of the branch to a pump device; a second fastener positioned on the lower portion of the extension strap above the thigh band and below the first fastener, the second fastener being configured to engage the catheter above the outlet end of the catheter and below the branch of the catheter; whereby the urine bag suspension system is free of fasteners above the branch in the catheter, such that disengaging the first and second fasteners positioned below the branch provides for disassembly of the suspension system from the catheter for cleaning purposes.

5. The urine bag suspension system according to claim 4 further comprising an anti-slip element disposed on the inside of at least one of the waist band and the thigh band.

6. The urine bag suspension system according to claim 5 wherein the anti-slip element comprises silicone material.

7. A urine bag suspension system for carrying a urine collection device including a catheter and a urine bag, the catheter having an engaging outlet end removably engaging the urine bag, and a branch depending therefrom providing for attachment of the branch to a pump device, the urine bag suspension system comprising: a waist band; a urine bag support suspended from the waist band for carrying a urine bag, wherein the urine bag support comprises an extension strap and a thigh band attached to the extension strap, the extension strap having an adjustment fastener on a portion of the surface of thereof, the adjustment fastener adapted to engage another portion of the surface of the extension strap, to form an S-fold therein, and thereby adjust the effective length of the extension strap, the thigh band being positioned on the extension strap relative to the waist band so as to wrap around the thigh of a patient; a pair of securing straps attached to the thigh band and the extension strap, each securing strap comprising a fastener portion that is configured to be received through an opening in a top corner of a urine collection bag, and to engage a mating portion on the thigh band, to thereby secure the urine bag to the suspension system; and a first fastener positioned on the extension strap above the thigh band, the first fastener being configured to engage the catheter below the branch only, to secure the catheter without securing the branch such that the first fastener resists downward movement of the catheter and provides for attachment of the branch to a pump device; a second fastener positioned on the extension strap above the thigh band and below the first fastener, the second fastener being configured to engage the catheter above the outlet end of the catheter and below the branch of the catheter; whereby the urine bag suspension system is free of fasteners above the branch in the catheter, such that disengaging the first and second fasteners positioned below the branch provides for disassembly of the suspension system from the catheter for cleaning purposes.

8. The urine bag suspension system according to claim 7 wherein the surface of the suspension strap has plurality of spaced indicators thereon, for indicating the effective length of the support strap, when the edge of the S-fold is aligned therewith.

9. The urine bag suspension system according to claim 8 wherein the adjustment fastener is positioned intermediate the ends of the suspension strap, and wherein the thigh strap is on the portion of the suspension strap between the adjustment fastener and the free end.

10. The urine bag suspension system according to claim 7 further comprising an anti-slip element disposed on the inside of at least one of the waist band and the thigh band.

11. The urine bag suspension system according to claim 10 wherein the anti-slip element comprises silicone material.
